(19) United States
(12) Patent Application Publication
(10) Pub. No.: US 2026/0295252 A1
(43) Pub. Date: Oct. 1, 2026

(54) FOLDABLE IMPLANTABLE MEDICAL DEVICE

(71) Applicant: Cochlear Limited, Macquarie University, NSW (AU)

(72) Inventors: Wim BERVOETS, Wilrijk (BE); Koen Erik VAN DEN HEUVEL, Hove (BE); Stijn EECKHOUDT, Keerbergen (BE)

(21) Appl. No.: 19/478,961

(22) PCT Filed: Jun. 5, 2024
(86) PCT No.: PCT/IB2024/055513

§ 371 Date: Oct. 27, 2025

Related U.S. Application Data
(60) Provisional application No. 63/507,861, filed on Jun. 13, 2023.

(51) Int. Cl. A61N 1/36

(52) U.S. Cl. CPC A61N 1/36036; A61N 1/361; A61N 1/36128

(57) ABSTRACT

Presented herein is a foldable medical device that is configured to be implanted in a tympanic cavity of recipient. More specifically, the medical device has a folded configuration with physical dimensions (physical footprint occupied by the device)/first form factor that enables insertion of the device through an ear canal of the recipient (e.g., through an opening formed at a tympanic membrane of the recipient). After insertion of the device into the tympanic cavity, the medical device is deployed from the folded configuration to an expanded configuration. In the expanded configuration, the medical device has physical dimensions (physical footprint occupied by the device)/second form factor that are larger than the physical dimensions in the folded configuration. In certain examples, the medical device is configured to be positioned against a promontory of the recipient's ear adjacent to the tympanic cavity.

### BACKGROUND

### Field of the Invention

The present invention relates generally to implantable medical devices.

### Related Art

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a medical device is provided. The medical device includes a base member configured to be implanted within a tympanic cavity of an ear of a recipient via an opening in or around a tympanic membrane of the recipient and at least one electrode disposed on the base member. The base member is configured to secure to a promontory of the ear within the tympanic cavity of the recipient, and the at least one electrode is configured for delivery of stimulation signals to the recipient. The medical device has a folded configuration for implantation into the tympanic cavity via the opening in or around the tympanic membrane and an expanded configuration for securement to the promontory.

In another aspect, a method is provided. The method includes forming an opening in or around a tympanic membrane of an ear of a recipient, inserting a medical device into a tympanic cavity of the ear via the opening, the medical device having a folded configuration during insertion through the opening, releasing the medical device to an expanded configuration after insertion of the medical device into the tympanic cavity, and securing the medical device, in the expanded configuration, against a promontory of the ear.

In yet another aspect, a medical device is provided. The medical device includes a resiliently flexible coil having a first configuration with a first form factor for insertion into a middle ear cavity of a recipient and a second configuration with a second form factor for deployment in the middle ear cavity. The medical device also includes at least one stimulating electrode and a stimulator unit electrically connected to the resiliently flexible coil and the at least one stimulating electrode. The stimulator unit is configured to generate electrical stimulation signals for delivery to the recipient via the at least one stimulating electrode.

### BRIEF DESCRIPTION OF THE DRAWINGS

Embodiments of the present invention are described herein in conjunction with the accompanying drawings, in which:

FIG. 1 is a schematic diagram illustrating an implantable medical device system with which aspects of the techniques presented herein can be implemented;

FIG. 2 is a block diagram of the implantable medical device system of FIG. 1;

FIGS. 3A, 3B, and 3C are perspective views of an implantable component of an implantable medical device system in different configurations, in accordance with certain embodiments presented herein;

FIG. 4 is a front view of another implantable component of an implantable medical device system, in accordance with certain embodiments presented herein;

FIG. 5 is a front view of yet another implantable component of an implantable medical device system, in accordance with certain embodiments presented herein;

FIG. 6 is a flowchart of a method of implanting an implantable medical device, in accordance with certain embodiments presented herein; and

FIG. 7 is a schematic diagram illustrating one example method for implanting an implantable medical device, in accordance with certain embodiments presented herein.

### DETAILED DESCRIPTION

Presented herein is a foldable medical device that is configured to be implanted in a tympanic cavity (middle ear cavity) of recipient. More specifically, the medical device has a folded configuration with physical dimensions (physical footprint occupied by the device)/first form factor that enables insertion of the device through an ear canal of the recipient (e.g., through an opening formed in or around a tympanic membrane of the recipient). After insertion of the device into the tympanic cavity, the medical device is deployed from the folded configuration to an expanded configuration. In the expanded configuration, the medical device has physical dimensions (physical footprint occupied by the device)/second form factor that are larger than the physical dimensions in the folded configuration. In certain examples, the medical device is configured to be positioned against a promontory of the recipient's ear adjacent to the tympanic cavity.

In some embodiments, the medical device is at least partially formed from a resiliently flexible material that enables deformation of the device to fold into the folded configuration via an applied force (e.g., a manually applied force). The resiliently flexible material has spring-like properties that, absent the applied force, bias transformation of the device toward the expanded configuration. For this reason, the device can be readily arranged in the folded configuration for insertion into the recipient and then accept the expanded configuration for deployment within the tympanic cavity. The foldability of the device facilitates greater ease of implantation in the recipient (e.g., via the ear canal, which has a relatively small opening), such as without having to form larger sized openings within the recipient (e.g., through the skull of the recipient) to enable the device to be inserted into the tympanic cavity.

There are a number of different types of devices in/with which embodiments of the present invention may be implemented. Merely for ease of description, the techniques presented herein are primarily described with reference to a specific device in the form of a hearing device configured to mitigate tinnitus symptoms of the recipient. However, it is to be appreciated that the techniques presented herein may also be partially or fully implemented by any of a number of different types of devices, including consumer electronic device (e.g., mobile phones), wearable devices (e.g., smartwatches), hearing devices, implantable medical devices, wearable devices, etc. consumer electronic devices, etc. As used herein, the term “hearing device” is to be broadly construed as any “device that acts on an actual or potential auditory perception of an individual, including to improve perception of sound signals, to reduce perception of sound signals, etc. In particular, a hearing device can deliver sound signals to a user in any form, including in the form of acoustical stimulation, mechanical stimulation, electrical stimulation, etc., and/or can operate to suppress all or some sound signals. As such, a hearing device can be a device for use by a hearing-impaired person (e.g., hearing aids, middle ear auditory prostheses, bone conduction devices, direct acoustic stimulators, electro-acoustic hearing prostheses, auditory brainstem stimulators, bimodal hearing prostheses, bilateral hearing prostheses, dedicated tinnitus therapy devices, tinnitus therapy device systems, combinations or variations thereof, etc.), a device for use by a person with normal hearing (e.g., consumer devices that provide audio streaming, consumer headphones, earphones, and other listening devices), a hearing protection device, etc. In other examples, the techniques presented herein can be implemented by, or used in conjunction with, various implantable medical devices, such as vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc. Indeed, even though the present disclosure primarily discusses implementations related to tinnitus, the techniques discussed herein, including the foldable implantable device, can be used in any suitable implementation, such as to deliver stimulations related to the ear (e.g., vestibular stimulations for maintaining balance).

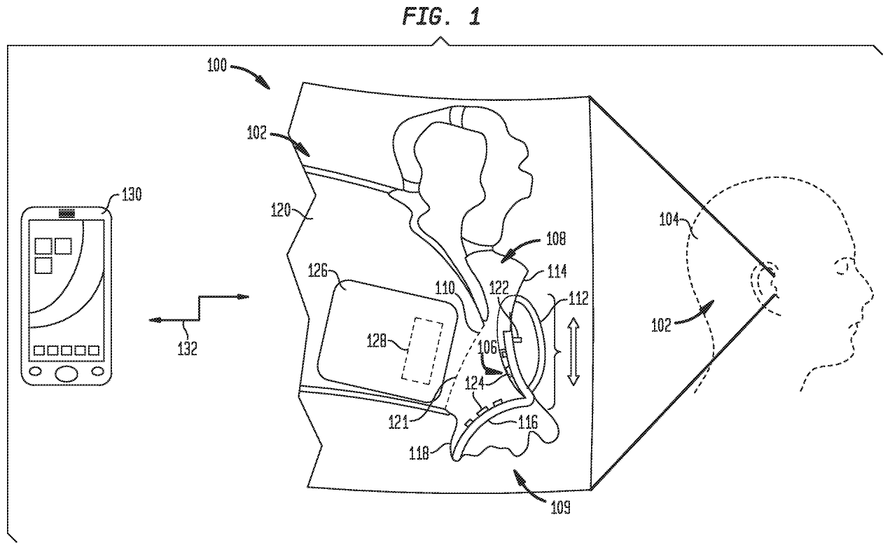

FIG. 1 is an example implantable medical device system (e.g., hearing device system) 100 with which aspects of the techniques presented herein can be implemented. The implantable medical device system 100 includes an implantable medical device (implantable component) 106 and an external device (external component) 126. The implantable component 106 is configured to be positioned within a tympanic cavity 108 (e.g., a middle ear cavity) in a middle ear 109 of a recipient. As such, the tympanic cavity 108 is located distal to a tympanic membrane 110 of the ear 102. The implantable component 106 includes a base member 112, which is configured to secure to a promontory 114 of the ear 102 within the tympanic cavity 108 (e.g., in a hypotympanum area, in a mesotympanum area, etc.) and, accordingly, fix the implantable component 106 within the tympanic cavity 108.

In the illustrated embodiment, the implantable component 106 also includes an extension member 116 that extends from the base member 112. In this example, each of the base member 112 and the extension member 116 is at least partially formed from a resiliently flexible material, such as a metal and/or a polymer (e.g., rubber, silicone), that has sufficient spring-like characteristics to bias the base member 112 and the extension member 116 away from one another toward an expanded configuration. For example, the implantable component 106 could be composed of a malleable wireframe structure. The biasing of the base member 112 and of the extension member 116 away from one another causes the extension member 116 to interact with a surface of the tympanic cavity 108 to bias the base member 112 against the promontory 114.

In one example, the extension member 116 is configured to abut against a tympanic wall 118 facing the promontory 114 within the tympanic cavity 108 (e.g., at a hypotympanum area of the tympanic cavity 108). For instance, the tympanic wall 118 defines or is part of a recess within the middle ear 109, and the extension member 116 is positioned within the recess and against the tympanic wall 118. The interaction (e.g., a frictional interface) between the extension member 116 and the surface of the tympanic wall 118 blocks movement of the extension member 116 relative to the tympanic wall 118. Similarly, the interaction between the base member 112 and the surface of the promontory 114 blocks movement of the base member 112 relative to the promontory 114. In this way, the implantable component 106 is wedged between the promontory 114 and the tympanic wall 118 to secure the implantable component 106 in the tympanic cavity 108.

In additional or alternative embodiments, the implantable component 106 is secured to the promontory 114 in another manner. As an example, an additional component or feature, such as an adhesive and/or a fastener, is applied to fix the implantable component 106 to the surface of the promontory 114. As another example, the base member 112 is large enough such that the surrounding tissue of the recipient 104 within the tympanic cavity 108 captures the base member 112 to block movement of the base member 112, thereby securing the base member 112 relative to the promontory 114. In such embodiments, the implantable component 106 may not have the extension member 116.

The resiliently flexible material of the base member 112 also enables the base member 112 to capture a contour of the promontory 114. That is, the base member 112 is sufficiently pliable such that, when pressed against the surface of the promontory 114, the base member 112 can bend to align with the contour of the promontory 114. In this manner, the base member 112 extends along and/or is flush with the surface of the promontory 114. For example, the base member 112 has a convex configuration when placed against the promontory 114 in the expanded configuration to conform to the promontory 114 and to avoid encroachment near the tympanic membrane 110. Such positioning of the base member 112 against the promontory 114 can further facilitate securement/fixation of the base member 112 to the promontory 114.

Additionally, the resiliently flexible material of the implantable component 106 enables the implantable component 106 to fold toward a folded configuration in response to an applied force (e.g., a manually applied force). For example, in the folded configuration, the extension member 116 is folded onto the base member 112 such that the base member 112 and the extension member 116 abut against one another and/or extend alongside one another. In some embodiments, in the folded configuration, a first portion of the base member 112 is also folded onto a second portion of the base member 112. As such, the implantable component 106 has a reduced size in the folded configuration (relative to the expanded configuration).

The folded configuration of the implantable component 106 facilitates insertion of the implantable component 106 into the ear 102 of the recipient 104. By way of example, in the folded configuration, the implantable component 106 has a form factor that enables positioning of the implantable component 106 within an ear canal 120, which is located exterior to the tympanic membrane 110. For instance, a dimension (e.g., a width, a thickness) of the implantable component 106 in the folded configuration is less than a diameter of the ear canal 120. Thus, in the folded configuration, the implantable component 106 can be inserted through the ear canal 120 and toward the tympanic cavity 108. To enable access to the tympanic cavity 108 from the ear canal 120, an opening 121 (shown in phantom lines) is formed through or around the tympanic membrane 110, and the opening 121 can accommodate the form factor of the implantable component 106 in the folded configuration to enable the implantable component 106 to be inserted from the ear canal 120 into the tympanic cavity 108 via the opening 121 of the tympanic membrane 110. As an example, the opening 121 may be formed by making an incision directly into the tympanic membrane 110. As another example, the opening 121 may be formed by making an incision adjacent to the tympanic membrane 110, such as around an outer boundary of the tympanic membrane 110 (e.g., to provide a tympanomeatal flap that can be moved to expose the opening 121).

The resiliently flexible material of the implantable component 106 enables the implantable component 106 to readily transition from the folded configuration to the expanded configuration absent an applied force that maintains the implantable component 106 in the folded configuration. In other words, the implantable component 106 is released from the folded configuration to the implantable component 106 absent any force imparted onto the implantable component 106. In additional or alternative embodiments, an additional force is applied to transition the implantable component 106 to and/or to maintain the implantable component in the expanded configuration. In further embodiments, the implantable component 106 transitions from the folded configuration to the expanded configuration in a different manner, such as upon exposure to an elevated temperature, upon receiving an electrical stimulant, in response to a chemical reaction, and so forth. In any of these cases, a user (e.g., a surgeon) can insert the implantable component 106 into the tympanic cavity 108 of the recipient 104 while the implantable component 106 is in the folded configuration, transition the implantable component 106 from the folded configuration to the expanded configuration after insertion of the implantable component 106 into the tympanic cavity 108, and deploy the implantable component 106 in the tympanic cavity 108 to secure to the promontory 114 while the implantable component 106 is in the expanded configuration.

In operation (e.g., while secured to the promontory 114), the implantable component 106 is configured to deliver electrical stimulation signals (stimulation) to the recipient. The stimulation signals (e.g., current) can have a number of different forms to provide different benefits to the recipient, such as to mitigate tinnitus effects, to enable perception of sounds, to compensate for balance dysfunction, to treat motor disorders (e.g., treat Parkinson's disease or ataxia), etc. To this end, the implantable component 106 includes one or more stimulation electrodes 122 (e.g., electrical stimulating contacts) that deliver electrical stimulation to the recipient. The stimulation electrode(s) 122 are positioned on the base member 112. The base member 112 is positioned within the tympanic cavity 108 such that the stimulation electrode(s) 122 are at an appropriate position to provide stimulation signals. For example, in the arrangement of FIG. 1, the base member 112 is positioned on the promontory 114 such that the stimulation electrode(s) 122 abut (are positioned against) the promontory 114. This location places the stimulation electrode(s) 122 within a threshold distance of the inner ear. Such a position of the stimulation electrode(s) 122 enables the stimulation electrode(s) 122 to deliver stimulation signals efficiently to the inner ear.

It should be noted that the implantable component 106 provides the electrical stimulation to the recipient from within the tympanic cavity 108, and not from within the inner ear of the recipient. As such, the implantable component 106 can be readily implanted within the recipient 104 and/or removed from the recipient 104. Additionally, the implantable component 106 can be manufactured separately and independently from the other components that may be implanted within the recipient 104. In this manner, a cost of manufacture of the implantable component 106 can be reduced, such as in comparison to a cost of manufacture of an embodiment in which an implantable component is a part of or integral to another component implanted within the recipient 104.

The implantable component 106 includes circuitry 124 (e.g., electrical components, processing circuitry). The circuitry 124 is configured to output control signals to cause the stimulation electrode(s) 122 to deliver the stimulation signals. The circuitry 124 can be at least partially enclosed by the base member 112 and the extension member 116. The circuitry 124 additionally or alternatively extends at least partially external to the base member 112 and/or the extension member 116, such as toward the tympanic membrane 110 while the implantable component 106 is deployed in the tympanic cavity 108 In some embodiments, the circuitry 124 is disposed on or in a flexible material to facilitate transition of the implantable component 106 between the folded configuration and the expanded configuration. Additionally or alternatively, the circuitry 124 is enclosed within one of the base member 112 or the extension member 116. Thus, the circuitry 124 may not be folded while the extension member 116 is moved relative to the base member 112. In some embodiments, the implantable component 106 is configured to output control signals without communicating with another component or device separate from the implantable component 106 via the circuitry 124. That is, at least for a period of time, the implantable component 106 can independently operate to deliver the stimulation signals.

In additional or alternative embodiments, the implantable component 106 can communicate with an external component 126 of the implantable medical device system 100 to deliver the stimulation signals based on communication with the external component 126. For instance, the external component 126 transmits data to the implantable component 106, and the stimulation electrode(s) 122 deliver the stimulation signals based on the data received from the external component 126. In the illustrated embodiment, the external component 126 is positioned within the ear canal 120 of the recipient 104. For instance, the external component 126 is sized so that tissue (e.g., a surrounding wall of the ear canal 120) of the recipient 104 within the ear canal 120 captures the external component 126 to retain the position of the external component 126 within the ear canal 120 (e.g., adjacent to the tympanic membrane 110 and to the implantable component 106).

In certain examples, one or more additional features can be used to secure the external component 126 within the ear canal 120. As an example, each of the implantable component 106 and the external component 126 includes a magnetic feature that enables the implantable component 106 and the external component 126 to magnetically engage one another. As another example, a temporary adhesive or a fastener is used to fix the external component 126 within the ear canal 120. The position of the external component 126 within the ear canal 120 and proximate to the implantable component 106 establishes a link (e.g., a radio frequency (RF) link) between the external component 126 and the implantable component 106 for communication.

The external component 126 transcutaneously transmits data and/or power to the implantable component 106 across the tympanic membrane 110. For instance, the link established between the external component 126 and the implantable component 106 enables the external component 126 to wirelessly transmit data to the implantable component 106 for delivering stimulation signals. To this end, the external component 126 includes an external coil 128, which is configured to transmit the data to the implantable component 106. For example, the implantable component 106 includes a corresponding implantable coil (e.g., integrated in a main body, a coil, or a housing) configured to receive the data transmitted by the external coil 128, and the implantable coil transmits the data to the circuitry 124 to cause the stimulation electrode(s) 122 to deliver stimulation signals.

The external component 126 can additionally or alternatively deliver power to the implantable component 106. That is, the implantable component 106 receives power via the link established between the external component 126 and the implantable component 106, and the implantable component 106 operates using the received power. For example, the circuitry 124 uses the power to operate the stimulation electrode(s) 122 to deliver stimulation signals.

In the illustrated embodiment, the implantable medical device system 100 is also able to communicate with a computing device 130, such as a personal computer (e.g., laptop, desktop, tablet), a mobile phone (e.g., smartphone), a hand-held device (e.g., a tablet computer), a surgical system, a remote control unit, a hearing aid or other implant device, etc. The computing device 130 and the implantable medical device system 100 wirelessly communicate via a bi-directional communication link 132. The bi-directional communication link 132 may include, for example, a short-range communication, such as a Bluetooth link, a Bluetooth Low Energy (BLE) link, a proprietary link, etc. As an example, the computing device 130 can transmit signals (e.g., control signals, data signals, power signals) via the bi-directional communication link 132 to cause the implantable component 106 and/or the external component 126 to operate. As another example, the implantable component 106 and/or the external component 126 can transmit signals to the computing device 130 to provide operational information to the computing device 130, which can then provide a notification (e.g., a display) to a user to indicate the operational information. Thus, the computing device 130 can further enhance operation of the implantable medical device system 100.

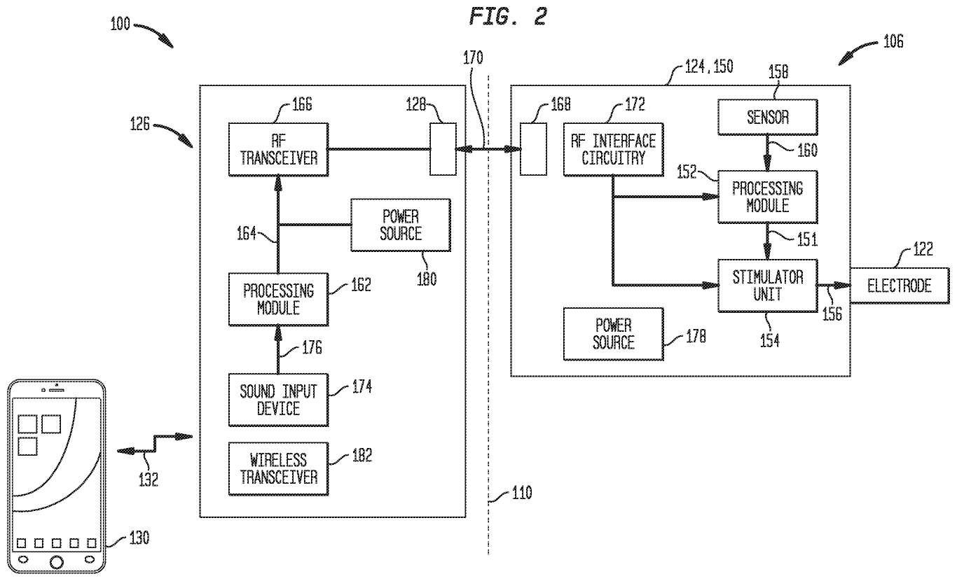

FIG. 2 is a block diagram of the implantable medical device system 100 including the implantable component 106 and the external component 126. In the illustrated embodiment, the implantable component 106 includes a medium 150 to which various parts of the circuitry 124 of the implantable component 106 are coupled. In some embodiments, the medium 150 includes a printed circuit board (PCB) that electrically connects at least some of the parts of the circuitry 124 to one another. In additional or alternative embodiments, the medium 150 includes an enclosure or housing in which the parts are disposed, and the medium 150 shields the parts from external elements (e.g., dust, debris). The medium 150 can extend along at least a portion of the base member 112 and/or of the extension member 116.

As described elsewhere herein, the implantable component 106 can operate independently from the external component 126, for at least a period of time, to stimulate the recipient 104. By way of example, the implantable component 106 includes an implantable processing module 152 configured to provide control signals 151 to a stimulator unit 154 of the implantable component 106 without the implantable component 106 having to receive initial signals (e.g., data signals) from the external component 126. The stimulator unit 154 is then configured to utilize the control signals 151 to generate stimulation signals 156 for delivery to the recipient 104 via the stimulation electrode(s) 122. In certain embodiments, the implantable component 106 includes one or more sensors 158 configured to provide sensor data 160 to the implantable processing module 152, and the implantable processing module 152 is configured to generate and transmit the control signals 151 based on the sensor data 160. As an example, the sensor data 160 includes received sound signals (e.g., sounds provided by a surrounding or ambient environment), a period of time, a parameter (e.g., biometric data, movement data) related to the recipient 104, and/or any other suitable data that indicates whether the stimulation signals 156 are to be delivered to the recipient 104.

The implantable processing module 152 can then generate the control signals 151 to cause the stimulator unit 154 to generate the stimulation signals 156 (e.g., stimulation signals 156 having certain characteristics) for delivery to the recipient 104. In additional or alternative embodiments, the implantable processing module 152 can cause the stimulation electrode(s) 122 to deliver the stimulation signals 156 without usage of the sensor(s) 158. For instance, the implantable processing module 152 can cause the stimulation electrode(s) 122 to deliver the stimulation signals 156 in a pre-determined manner, such as to provide stimulation signals having pre-determined characteristics (e.g., to cause the recipient 104 to perceive pre-determined sound) regardless of signals in a surrounding environment.

As described elsewhere herein, the external component 126 is configured to deliver signals to the implantable component 106, such as transcutaneously across the tympanic membrane 110. The illustrated external component 126 includes an external processing module 162 configured to transmit control signals 164 directed to the implantable component 106 for delivering the stimulation signals 156. For example, the external processing module 162 outputs the control signals 164 to an RF transceiver 166 of the external component 126, and the RF transceiver 166 transcutaneously transfers the control signals 164 (e.g., in an encoded manner) to the implantable component 106 via the external coil 128. The implantable component 106 includes an implantable coil 168 (e.g., an inductive coil) disposed in the medium 150 and communicatively coupled to the external coil 128 via a wireless link 170 (e.g., an RF link, an infrared link, an electromagnetic link, a capacitive link, an inductive link) formed between the external coil 128 and the implantable coil 168.

The implantable coil 168 receives the control signals 164 from the external component 126 via the wireless link 170. The implantable component 106 further includes RF interface circuitry 172 configured to receive the control signals 164 via the implantable coil 168. The RF interface circuitry 172 is configured to transmit the control signals 164 to the implantable processing module 152, which then processes the control signals 164 to transmit the control signals 151 to the stimulator unit 154 for delivering the stimulation signals 156. For example, the control signals 164 includes less processed information, and the implantable processing module 152 can perform processing operations to convert the control signals 164 to the control signals 151 usable by the stimulator unit 154. Additionally or alternatively, the RF interface circuitry 172 is configured to deliver the control signals 164 directly to the stimulator unit 154, bypassing the implantable processing module 152, and the stimulator unit 154 is configured to generate and transmit the stimulation signals 156 based on the control signals 164 received from the RF interface circuitry 172.

Each of the implantable processing module 152 and the external processing module 162 may include, for example, one or more processors and a memory device (memory) that includes sound processing logic. The memory device is one or more software or hardware-based computer-readable storage media operable to store information accessible by the one or more processors, and the memory device can include any one or more of: Non-Volatile Memory (NVM), Ferroelectric Random Access Memory (FRAM), read only memory (ROM), random access memory (RAM), Electronically-Erasable Programmable Read-Only Memory (EEPROM), magnetic disk storage media devices, optical storage media devices, flash memory devices, electrical, optical, magnetic, solid state, or other physical/tangible memory storage devices. The memory device can include transitory memory or non-transitory memory. By way of example, the memory device can include wired media, such as a wired network or direct-wired connection, and/or wireless media, such as acoustic, RF, infrared, other wireless media, or combinations thereof. The one or more processors (e.g., microprocessors, microcontrollers) are, for example, hardware or software processors (e.g., Central Processing Units) that can obtain and execute instructions stored on the memory device. The one or more processors can also communicate with and control the operation of other parts of the implantable medical device system 100. For example, the one or more processors (e.g., processing element(s) implementing firmware, software, etc.) in the implantable processing module 152 execute instructions in the memory device of the implantable processing module 152 to generate and output the control signals 151, and the one or more processors in the external processing module 162 execute instructions stored in the memory device of the external processing module 162 to generate and output the control signals 164.

In certain embodiments, the external component 126 includes one or more sound input devices 174 (e.g., one or more external microphones, audio input ports, data ports, telecoils, etc.) configured to receive input audio signals 176 and transmit the input audio signals 176 to the external processing module 162. The external processing module 162 then processes the input audio signals 176 to generate and output the control signals 164 based on the input audio signals 176. For example, the external processing module 162 generates control signals 164 that would cause generation of stimulation signals 156 that enable the recipient 104 to perceive input audio captured by the sound input device(s) 174. Thus, the input audio signals 176 received by the sound input device(s) 174 are used as basis for delivering the stimulation signals 156.

The implantable component 106 includes an implantable power source 178 (e.g., one or more batteries, one or more capacitors, etc.), which provides power that enables operation of the implantable component 106 (e.g., the implantable processing module 152, the stimulator unit 154, the implantable sound processing module 158, the RF interface circuitry 172). The external component 126 is additionally or alternatively configured to provide power used to enable operation of the implantable component 106. For example, the implantable power source 178 is rechargeable, and the power provided by the external component 126 is delivered to the implantable power source 178. To this end, the external component 126 includes an external power source 180. The RF transceiver 166 receives power from the external power source 180 and transmits the power to the implantable coil 168 of the implantable component 106 via the external coil 128. The power source 178 receives the power from the implantable coil 168, and the power is available for usage by remaining parts of the circuitry 124 for operation.

The external component 126 further includes a wireless transceiver 182 that is communicatively coupled to the computing device 130 via the bi-directional communication link 132. For example, the computing device 130 can transmit data to the wireless transceiver 182 via the bi-directional communication link 132, and/or the wireless transceiver 182 can transmit data to the computing device 130 via the bi-directional communication link 132. Data communication between the external component 126 and the computing device 130 can effectuate certain operation of the external component 126, of the computing device 130, and/or of the implantable component 106. Although FIG. 2 illustrates the external processing module 162, the RF transceiver 166, the sound input device(s) 174, and the external power source 180 as being implemented at the external component 126, it is to be appreciated that these elements (e.g., functional operations) could also or alternatively be implemented as part of the computing device 130.

In some embodiments, the implantable component 106 does not include an implanted magnet and/or ferromagnetic materials. The absence of the implanted magnet can reduce complexity associated with maintaining the implantable component 106 within the recipient. By way of example, during a magnetic resonance imaging (MRI) operation or other situations in which magnetic fields are provided to the recipient, an entirety of the implantable component 106 can remain within the recipient, and the provided magnetic fields, which can otherwise cause undesirable movement and/or undesirably adjustment of an implanted magnet, do not affect the positioning of the implantable component 106.

As noted above, the implantable component 106 is foldable between a folded configuration and an expanded configuration, and vice versa. To this end, the medium 150 and the different parts of the implantable component 106, such as the implantable processing module 152, the stimulator unit 154, the one or more sensors 158, the implantable coil 168, the RF interface circuitry 172, the RF interface circuitry 172, and/or the implantable power source 178, are configured (e.g., positioned or spatially arranged) to enable folding of the implantable component 106. In this manner, different portions of the implantable component 106 are foldable to enable the implantable component 106 to fold in a desirable manner to facilitate insertion and deployment in the tympanic cavity 108.

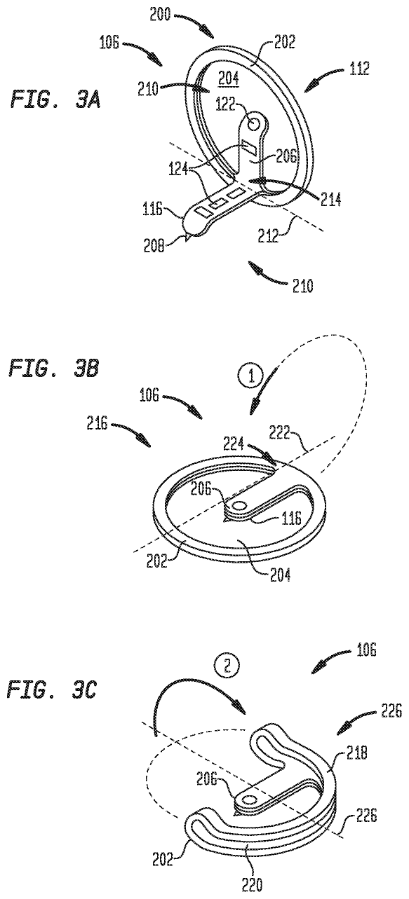

FIGS. 3A, 3B, and 3C are perspective views of the implantable component 106 in different configurations. More specifically, FIG. 3A illustrates an expanded configuration 200 (e.g., an unfolded configuration) for the implantable component in which the extension member 116 extends away from the base member 112. In this arrangement, the base member 112 has a circular shape with an annular main body 202 (e.g., a coil, a housing enclosing a coil) that defines a central opening 204, and the extension member 116 has an elongate shape extending from the annular main body 202 and away from the central opening 204 in the expanded configuration 200. In additional or alternative embodiments, the base member 112 has any other suitable shape (e.g., a triangular shape, a rectangular shape, an irregular shape) and/or the base member 112 does not define the central opening 204 (e.g., the base member 112 includes a continuous or solid shape) to accommodate other parts (e.g., the circuitry 124, the medium 150) of the implantable component 106 and/or to provide sufficient flexibility of the base member 112.

The base member 112 also includes a central member 206, which has an elongate shape extending from the annular main body 202 and into the central opening 204. As an example, the extension member 116 and the central member 206 are circumferentially aligned with one another about the annular main body 202. In additional or alternative embodiments, the extension member 116 and the central member 206 are circumferentially offset from one another.

The circuitry 124 extends from the central member 206 to the extension member 116. Thus, in the expanded configuration 200, the circuitry 124 is generally unfolded, and the circuitry 124 extends across at least a portion of the annular main body 202. Additionally, the stimulation electrode(s) 122 are positioned at a distal end 210 of the central member 206 within the central opening 204. As such, the central member 206 can be exposed and/or uninsulated to facilitate delivery of electrical stimulation via the stimulation electrode(s) 122. Moreover, in this specific example, the implantable component 106 includes one or more return electrodes 208 positioned at a distal end 210 of the extension member 116 external to the central opening 204 in the expanded configuration 200. The circuitry 124 extends along each of the central member 106 and the extension member 116 to electrically couple to the stimulation electrode(s) 122 and to the return electrode(s) 208. As such, the circuitry 124 can operate the electrodes 122, 208 (e.g., deliver electrical flow to the electrodes 122, 208) to enable the stimulation electrode(s) 122 to deliver stimulation to the recipient 104.

The resiliently flexible material of the implantable component 106 enables the extension member 116 and the annular main body 202 to be folded about one another such that the extension member 116 extends alongside and/or abuts against the central member 206. For instance, the extension member 116 and the annular main body 202 can rotate relative to one another about a first axis 212 (shown in FIG. 3A) that extends along a junction 214 at which the extension member 116 extends from the annular main body 202. As shown in FIG. 3B, folding of the extension member 116 and the annular main body 202 relative to one another about the first axis 212 transitions the implantable component 106 from the expanded configuration 200 to a first folded configuration 216 (e.g., a partially folded configuration) in which the extension member 116 extends into or in radial overlap with the central opening 204.

The implantable component 106 is relatively smaller in size in the first folded configuration 216 than in the expanded configuration 200. That is, the implantable component 106 has a first form factor in the expanded configuration 200, the implantable component 106 has a second form factor in the first folded configuration 216, and the second form factor occupies a smaller physical footprint as compared to the first form factor. By way of example, the outer boundary of the implantable component 106 in the first folded configuration 216 includes the perimeter (e.g., circumference) of the annular main body 202, whereas the outer boundary of the implantable component 106 in the expanded configuration 200 includes the perimeter of the annular main body 202 plus the perimeter of the extension member 116.

In the example of FIGS. 3A-3C, the resiliently flexible material of the implantable component 106 further enables folding of different portions of the annular main body 202 relative to one another. As an example, a first portion 218 (e.g., a first half, a first semicircular portion) of the annular main body 202 and a second portion 220 (e.g., a second half, a second semicircular portion) of the annular main body 202 can rotate relative to one another about a second axis 222 (shown in FIG. 3B) that extends along the central member 206 and at a junction 224 at which the first portion 218 and the second portion 220 are joined. As shown in FIG. 3C, folding of the first portion 218 of the annular main body 202 and the second portion 220 of the annular main body 202 relative to one another about the second axis 222 transitions the implantable component 106 from the first folded configuration 216 to a second folded configuration 226 (e.g., a fully folded configuration) in which the first portion 218 of the annular main body 202 and the second portion 220 of the annular main body 202 extend alongside and/or abut one another.

The implantable component 106 is even smaller sized in the second folded configuration 226 than in the first folded configuration 216. For example, the implantable component 106 has a third form factor in the second folded configuration 226, and the third form factor occupies a smaller physical footprint as compared to each of the first form factor associated with the expanded configuration 200 and the second form factor associated with the first folded configuration 216. For instance, the outer boundary of the implantable component 106 in the second folded configuration 226 includes the perimeter of one of the first portion 218 of the annular main body 202 or the second portion 220 of the annular main body 202, rather than the perimeter of the entire annular main body 202.

The third form factor of the implantable component 106 facilitates insertion of the implantable component 106 in the tympanic cavity 108. For instance, the size and/or shape of the third form factor enables movement of the implantable component 106 through the ear canal 120 (e.g., a dimension of the third form factor is smaller than a diameter of the ear canal 120), into the opening 121 formed through or around the tympanic membrane 110, and into the tympanic cavity 108. After insertion of the implantable component 106 in the tympanic cavity 108, the implantable component 106 can be released to transition to the expanded configuration 200. As an example, a force is applied to transition the implantable component 106 to and maintain the implantable component 106 in the second folded configuration 226. The force may be removed after the implantable component 106 is inserted into the tympanic cavity 108. Upon removing the force, the resiliently flexible material of the implantable component 106 urges/biases the implantable component 106 toward the expanded configuration 200. As another example, the implantable component 106 is formed from a shape memory alloy in which the implantable component 106 is deformable while exposed to a temperature below a threshold and the implantable component 106 returns to a pre-deformed configuration when exposed to a temperature above the threshold. For instance, the expanded configuration 200 is the pre-deformed configuration of the implantable component 106, and the implantable component 106 is deformed to and retained in the second folded configuration 226 prior to insertion in the recipient. Upon insertion in the recipient, the implantable component 106 is exposed to a higher temperature (e.g., a body temperature of the recipient, temperature emitted from a separate heating device) to cause the implantable component 106 to transition from the second folded configuration 226 to the pre-deformed expanded configuration 200. As such, the implantable component 106 can remain in the second folded configuration 226 without having to sustain application of a force, and the implantable component 106 readily transitions from the second folded configuration 226 to the expanded configuration 200 upon insertion. In either case, the first portion 218 of the annular main body 202 rotates about the second axis 222 away from the second portion 220 of the annular main body 202 to transition the implantable component 106 toward the first folded configuration 216, and the extension member 116 rotates about the first axis 212 away from the central member 206 to transition the implantable component 106 toward the expanded configuration 200. As such, the implantable component 106 is in the expanded configuration 200 in the tympanic cavity 108.

The first form factor of the implantable component 106 in the expanded configuration 200 facilitates deployment of the implantable component 106 in the tympanic cavity 108. For example, in the expanded configuration 200, the base member 112 is shaped (e.g., has a convex configuration) to facilitate securement to the promontory 114. Moreover, in the expanded configuration 200, the extension member 116 extends obliquely relative to the base member 112 (e.g., away from the annular main body 202) to facilitate abutment against the tympanic wall 118. Thus, the expanded configuration 200 facilitates fixation/securement of the implantable component 106 within the tympanic cavity 108.

In the illustrated embodiment, the central member 206 is not folded to transition the implantable component 106 to either the first folded configuration 216 or the second folded configuration 226. For example, the central member 206 is included within the outer boundary of the implantable component 106 in each of the expanded configuration 200, the first folded configuration 216, and the second folded configuration 226. Therefore, folding of the central member 206 may not reduce the size of the implantable component 106. However, in additional or alternative embodiments, the central member 206 can be folded.

Indeed, the implantable component 106 can be folded in any suitable manner to reduce a size of the implantable component 106. By way of example, from the second folded configuration 226, part of the first portion 218 of the annular main body 202, part of the second portion 220 of the annular main body 202, and/or part of the central member 206 are configured to fold about a third axis 226, which bisects the annular main body 202. Such folding of the implantable component 106 from the second folded configuration 226 further reduces a size of the implantable component 106 (e.g., to halve the size as compared to the second folded configuration 226).

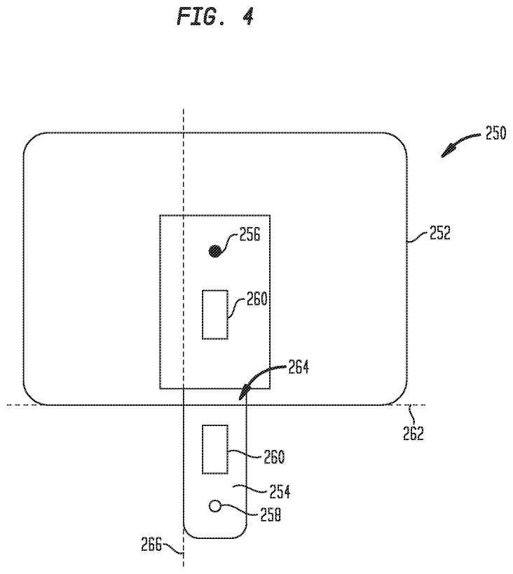

FIG. 4 is a front view of an implantable component 250, in accordance with certain embodiments presented. The illustrated implantable component 250 includes a main body 252 having a rectangular shape, as well as an extension member 254 having an elongate shape. The main body 252 has a continuous or solid shape and therefore does not define any openings. Thus, the implantable component 250 does not include a member similar to the central member 206 of the implantable component 106. Instead, one or more stimulation electrode(s) 256 of the implantable component 250 are positioned directly on a surface of the main body 252 (e.g., at a central location on the main body 252). Additionally, one or more return electrode(s) 258 of the implantable component 250 are positioned on the extension member 254. The implantable component 250 also includes circuitry 260, which extends from the extension member 254 to the main body 252 to electrically connect to the electrodes 256, 258.

The implantable component 250 is composed of a resiliently flexible material and is configured to fold (e.g., via an applied force). As an example, the main body 252 and the extension member 254 are configured to rotate relative to one another about a first axis 262 extending along a junction 264 at which the extension member 254 extends from the main body 252. As another example, different portions of the main body 252 are configured to rotate relative to one another about a second axis 266 that extends alongside the extension member 254. The rotation of such parts of the implantable component 250 transitions the implantable component 250 to a folded configuration (e.g., a fully folded configuration) to reduce a size of the implantable component 250. The folded configuration of the implantable component 250 can facilitate insertion of the implantable component 250 into the tympanic cavity 108 of the recipient 104, such as positioning and movement through the ear canal 120 and through the opening 121 formed into or around the tympanic membrane 110. Additionally, the resiliently flexible material of the implantable component 250 urges transition of the implantable component 250 to an expanded configuration, such as by urging rotation of the extension member 254 and the main body 252 away from one another about the first axis 262 and/or rotation of the portions of the main body 252 away from one another about the second axis 266, that facilitates deployment of the implantable component 250 in the tympanic cavity 108 (e.g., securement of the main body 252 to the promontory 114).

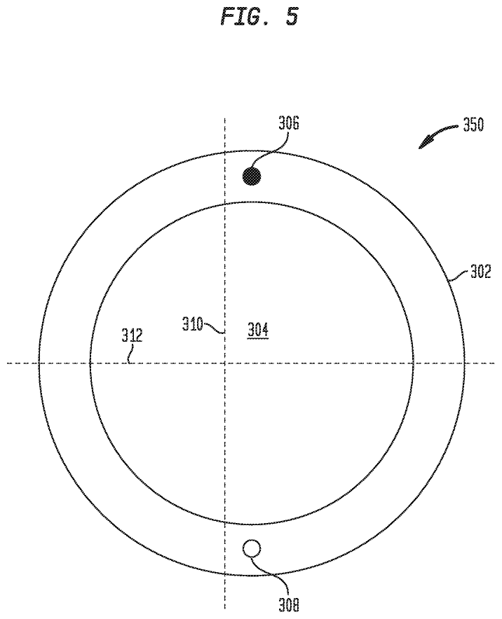

FIG. 5 is a front view of another implantable component 350 having an annular main body 302 that defines a central opening 304. However, the implantable component 350 does not include any members (e.g., extension members) extending from the main body 302 (e.g., toward or away from the central opening 304). Instead, one or more stimulation electrode(s) 306 and return electrode(s) 308 of the implantable component 300 are positioned directly on a surface of the annular main body 302. In such an embodiment, circuitry that is electrically coupled to the stimulation electrode(s) 306 and to the return electrode(s) 308 extends about at least a portion of the perimeter (e.g., the circumference) of the annular main body 302. Although the annular main body 302 includes a circular shape defining the central opening 304, in additional or alternative embodiments, the implantable component 350 can have a main body with any suitable shape (e.g., a rectangular shape, a triangular shape, an irregular shape) that may or may not define a central opening.

The implantable component 350 is also composed of a resiliently flexible material and is configured to fold (e.g., via an applied force). For example, different portions of the annular main body 302 are configured to rotate relative to one another about a first axis 310 to reduce a size of the implantable component 300, thereby facilitating insertion of the implantable component 300 into the tympanic cavity 108 of the recipient 104. In some embodiments, the annular main body 302 can be folded multiple times. For instance, after folding different portions of the annular main body 302 about the first axis 310, additional portions of the annular main body 302 can be folded about a second axis 312, further reducing the size of the implantable component 300. The resiliently flexible material of the implantable component 300 also urges transition of the implantable component 300 to an expanded configuration (e.g., rotation of the portions of the annular main body 302 away from one another about the first axis 310 and/or about the second axis 312) that facilitates deployment of the implantable component 300 in the tympanic cavity 108.

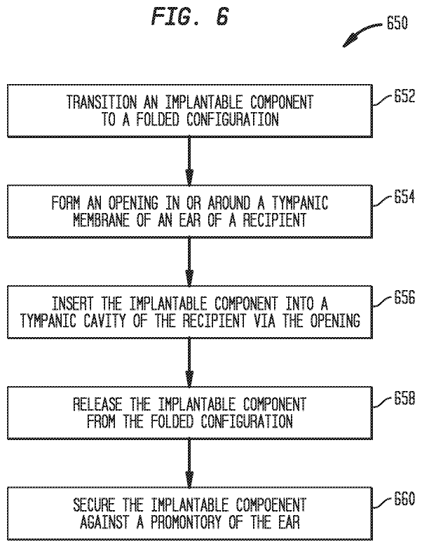

FIG. 6 is a flowchart of a method 650 of implanting any of the implantable components described herein into a recipient. The method 650 can be manually performed (e.g., by a surgeon) and/or automatically performed (e.g., by robotic surgical equipment), and or by combinations thereof. In some embodiments, the method 650 is performed by a single entity. Alternatively, different entities can perform different operations of the method 650. It should be noted that the method 650 can be performed differently than depicted in different embodiments. For example, a depicted operation may not be performed, an additional operation can be performed, and/or any of the depicted operations can be performed in a different order.

At 652, the implantable component is transitioned to a folded configuration. As an example, the implantable component includes a base member and an extension member extending from the base member. The base member and the extension member are rotated about one another such that the extension member extends within an outer boundary of the base member (e.g., the extension member extends along the base member) to reduce a size of the implantable component. As another example, different portions of the base member of the implantable component are rotated about one another such that the portions extend along one another to reduce the size of the implantable component. The implantable component is composed of a resiliently flexible material to enable a force to be applied to transition the implantable component to the folded configuration and/or to maintain the implantable component in the folded configuration. By way of example, forceps are used to grip the implantable component to apply the force that maintains the implantable component in the folded configuration. Additionally or alternatively, the implantable component is maintained in the folded configuration absent exposure to an elevated temperature.

At 654, an opening is formed in or around a tympanic membrane of an ear of the recipient. For example, an incision is made through the tympanic membrane or near an outer boundary of the tympanic membrane. As a result, a tympanic cavity of the recipient is exposed to an ear canal of the recipient via the opening at the tympanic membrane. In other words, the tympanic cavity is accessible from the ear canal via the opening at the tympanic membrane. By way of example, the opening is surgically formed to have a size that can accommodate the implantable component in the folded configuration.

At 656, the implantable component is inserted into the tympanic cavity of the recipient via the opening. For instance, a dimension (e.g., a diameter, a width, a thickness) of the implantable component in the folded configuration is less than a diameter of the ear canal (e.g., of the most narrow region of the ear canal) to enable positioning and movement of the implantable component within the ear canal, toward the opening at the tympanic cavity, and into the tympanic cavity via the opening at the tympanic membrane. In certain embodiments, the forceps used to maintain the implantable component in the folded configuration are also used to insert the implantable component into the tympanic cavity via the ear canal, such as with use of a speculum or catheter extending through the ear canal toward the tympanic cavity.

At 658, the implantable component is released from the folded configuration, thereby causing the implantable component to transition to an expanded configuration in the tympanic cavity. For instance, the force previously applied to maintain the implantable component in the folded configuration is removed. Additionally or alternatively, the implantable component is exposed to an elevated temperature (e.g., a body temperature of the recipient). As a result, the resiliently flexible material of the implantable component urges the implantable component to unfurl to the expanded configuration. For example, portions of the base member rotate about one another and/or the base member and the extension member rotate about one another to extend away from one another, thereby increasing the size of the implantable component.

At 660, the implantable component is secured against a promontory of the ear to enable a stimulation electrode attached to the base member to contact the promontory. For example, in the expanded configuration, the base member can readily secure to the promontory (e.g., the resiliently flexible material of the base member enables the base member to conform to a contour of the promontory). In some embodiments, the implantable component is wedged into the tympanic cavity in that the extension member interfaces with a surface in the tympanic cavity to bias the base member against the promontory. In additional or alternative embodiments, an additional feature, such as an adhesive and/or a fastener (e.g., applied on the base member adjacent to the stimulation electrode), is used to secure the base member to the promontory. In further embodiments, surrounding tissue within the tympanic cavity captures the base member to secure the base member to the promontory. In yet another embodiment, the implantable component is secured by inserting the stimulation electrode attached to the base member within a hole (e.g., having a diameter of 0.5 millimeters) formed (e.g., surgically formed) through the promontory via an interference fit. Thus, the promontory captures the electrode, thereby retaining the position of the electrode and of the base member within the tympanic cavity. After the implantable component is secured within the tympanic cavity, the tympanic membrane can be sealed to remove the opening, thereby shielding the interior of the ear of the recipient and the implantable component disposed therein.

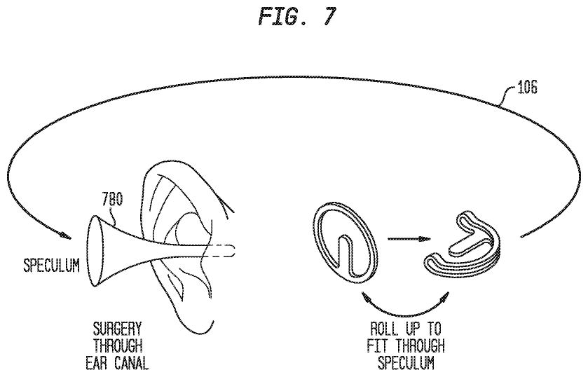

FIG. 7 is a schematic diagram illustrating one technique for implantation of an implantable medical device, such as implantable component 106 described above, in accordance with certain embodiments presented herein. In this example, the implantable component 106 is folded/rolled to a folded configuration that is configured to fit through a septum 780 positioned in the ear canal of a recipient.

The implantable component can be secured against the promontory at a target location that positions the stimulation electrode (e.g., one of the stimulation electrodes 122) in a desirable manner to provide stimulation signals to the recipient. For example, the target location of the implantable component positions the stimulation electrode within a threshold distance of a round window, of a round window niche, and/or of a cochlea of the recipient. Such a target location can cause at least a portion of the base member to which the stimulation electrode is attached to overlap with the round window and/or the round window niche. However, in some embodiments, securement of the implantable component to the promontory avoids applying direct pressure onto the round window to avoid affecting a structure and/or a function of the round window. The positioning of the implantable component within the tympanic cavity also positions a return electrode (e.g., the return electrode 208) of the implantable component in a desirable manner, such as in contact with tissue and/or bone of the recipient within the tympanic cavity. The target location of the implantable component can include a hypotympanum area and/or a mesotympanum area within the tympanic cavity.

It should be noted that operations similar to those described with respect to the method 650 can be performed to remove the implantable component from the recipient. For instance, an opening is formed in or around the tympanic membrane of the ear to enable access to the implantable component positioned within the tympanic cavity. Access to the implantable component via the opening at the tympanic membrane enables the implantable component to be decoupled from the promontory of the ear and transitioned from the expanded configuration to the folded configuration (e.g., via an applied force). Then, while in the folded configuration, the implantable component is moved from the tympanic cavity into the ear canal via the opening at the tympanic membrane, and then along the ear canal and out of the ear of the recipient.

Such operations described herein enables implantation and/or removal of the implantable component with respect to the recipient without having to perform more invasive or intensive operations. For example, a single opening is formed in the recipient (e.g., in or around the tympanic membrane) to implant and/or remove the implantable component, and the size of the opening is relatively small to accommodate the size of the implantable component in the folded configuration. Indeed, the foldability of the implantable component facilitates ease of implantation and/or removal of the implantable component, such as without having to form additional openings (e.g., in the skull of the recipient, through additional tissue of the recipient) and/or without having to form openings having a relatively larger size to accommodate movement of the implantable component therethrough. For this reason, implantation and/or removal of the implantable component is simplified or otherwise improved.

Moreover, the foldability and/or flexibility of the implantable component enables the implantable component to be readily implanted within any recipient. For example, the size of the implantable component in the folded configuration may be sufficiently small for insertion into the ear canal of any recipient (e.g., differently sized ear canals of different recipients). Additionally, the adjustability of the implantable component enables the implantable component to accommodate different anatomical characteristics (e.g., different contours of ear promontory) of different recipients. As such, the same embodiment of the implantable component can be inserted into and deployed within different recipients. Therefore, manufacture of different embodiments of the implantable component (e.g., a specific embodiment dedicated to a recipient with a particularly sized ear canal and/or having a promontory with a particular contour) can be avoided. In this manner, a cost and/or complexity associated with manufacture of the implantable component is reduced.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments may be combined with another in any of a number of different manners.

1. A medical device, comprising:
a base member configured to be implanted within a tympanic cavity of an ear of a recipient via an opening in or around a tympanic membrane of the recipient, wherein the base member is configured to secure to a promontory of the ear within the tympanic cavity; and
at least one electrode disposed on the base member, the at least one electrode being configured for delivery of stimulation signals to the recipient,
wherein the medical device has a folded configuration for implantation into the tympanic cavity via the opening in or around the tympanic membrane and an expanded configuration for securement to the promontory.

2. The medical device of claim 1, further comprising a resiliently flexible inductive coil disposed in the base member.

3. The medical device of claim 1, wherein the expanded configuration of the medical device comprises a convex configuration to capture a contour of the promontory of the ear.

4. The medical device of claim wherein the medical device comprises an extension member extending from the base member, and wherein, in the expanded configuration of the base member, the extension member is configured to interact with a surface in the tympanic cavity to bias the base member against the promontory.

5. The medical device of claim 4, wherein, in the expanded configuration of the base member, the extension member is configured to abut against a tympanic wall facing the promontory within the tympanic cavity.

6. The medical device of claim 4, further comprising at least one return electrode disposed at a distal end of the extension member.

7. The medical device of claim 4, wherein the extension member is configured to rotate relative to the base member to transition the medical device between the folded configuration and the expanded configuration.

8. The medical device of claim 1, wherein the base member comprises an annular main body defining a central opening.

9. The medical device of claim 8, wherein the medical device comprises a central member extending into the central opening, and wherein the at least one electrode is disposed on the central member.

10. The medical device of claim 8, wherein the at least one electrode is disposed on a surface of the annular main body.

11. The medical device of claim 1; further comprising:
a stimulator unit electrically connected to the at least one electrode, wherein the stimulator unit is configured to generate the stimulation signals for delivery to the recipient via the at least one electrode.

12. The medical device of claim 11, wherein the stimulator unit is configured to generate the stimulation signals for mitigation of tinnitus symptoms experienced by the recipient, for treatment of a motor disorder experienced by the recipient, or for treatment of balance dysfunction experienced by the recipient.

13. (canceled)

14. (canceled)

15. The medical device of claim 11, wherein the stimulator unit is configured to generate the stimulation signals based on data transmitted by an external component.

16. The medical device of claim 11, further comprising an implantable processing module, wherein the implantable processing module is configured to output control signals to the stimulator unit to cause the stimulator unit to generate the stimulation signals.

17. A method of implanting a medical device in a recipient, comprising:
forming an opening at a tympanic membrane of an ear of the recipient;
inserting the medical device into a tympanic cavity of the ear via the opening, wherein the medical device has a folded configuration during insertion through the opening;
after insertion of the medical device into the tympanic cavity, releasing the medical device to an expanded configuration; and
securing the medical device, in the expanded configuration, against a promontory of the ear.

18. The method of claim 17, comprising:
folding the medical device into the folded configuration prior to insertion through the opening at the tympanic membrane.

19. The method of claim 18, comprising:
applying a force to fold the medical device into the folded configuration; and
removing the force applied to fold the medical device to release the medical device to the expanded configuration.

20. The method of claim 18, comprising:
exposing the medical device to an elevated temperature after insertion of the medical device into the tympanic cavity to release the medical device to the expanded configuration.

21. The method of claim 17, wherein securing the medical device against the promontory comprises:
applying at least one of an adhesive or a fastener to the medical device.

22. The method of claim 17, wherein the medical device comprises a base member and an extension member extending from the base member, and wherein securing the medical device against the promontory comprises:
abutting the base member against the promontory of the ear; and
abutting the extension member against a tympanic wall facing the promontory within the tympanic cavity such that the base member is biased against the promontory.

23-28. (canceled)
